(19) United States
(12) Patent Application Publication
(10) Pub. No.: US 2015/0245778 A1
(43) Pub. Date: Sep. 3, 2015

(54) Low Power Perfusion Imaging

(71) Applicants: Siemens Aktiengesellschaft, Munchen (DE); Massachusetts General Hospital Corporation, Boston, MA (US)

(72) Inventors: Uvo Hölscher, Erlangen (DE); Keith Heberlein, Charlestown, MA (US); Ovidiu Andronesi, Melrose, MA (US)

(21) Appl. No.: 14/634,962

(22) Filed: Mar. 2, 2015

Related U.S. Application Data
(60) Provisional application No. 61/945,936, filed on Feb. 28, 2014.

(51) Int. Cl. A61B 5/026; A61B 5/00

(52) U.S. Cl. CPC A61B 5/0263; A61B 5/0042; A61B 2576/026; A61B 2560/0487

(57) ABSTRACT

Disclosed herein is a framework for facilitating low power perfusion imaging. In accordance with one aspect, a gradient-modulated constant-adiabaticity radio frequency pulse sequence is generated. A labeling pulse from the pulse sequence is applied to a region of interest to label fluid supplying the region of interest and to acquire a tagged image. A control pulse from the pulse sequence may also be applied to the region of interest to acquire a control image. A perfusion image may then be generated using the tagged image and the control image.

### CROSS-REFERENCE TO RELATED APPLICATION

This application claims the benefit of U.S. provisional application No. 61/945,936 filed on Feb. 28, 2014, the entire contents of which are incorporated herein by reference.

### TECHNICAL FIELD

The present disclosure generally relates to systems and methods for low power perfusion imaging.

### BACKGROUND

Magnetic resonance (MR) scanners use a large static magnetic field to align the nuclear spins of atoms as part of the procedure for producing images within the body of a patient. This large static magnetic field is referred to as the B₀ field. During an MR scan, Radio Frequency (RF) pulses generated by a transmitter coil cause perturbations to the local magnetic field, and RF signals emitted by the nuclear spins are detected by a receiver coil (or antenna). The transmitted RF field is referred to as the B₁ field. The RF signals are used to construct the MR images.

MR perfusion methods can be used to measure blood flow in different body regions. One set of MR methods uses injection of a contrast agent (e.g., Gadolinium) in the blood to measure wash-in and wash-out behavior in imaged slices of patient anatomy (e.g., brain). Another set of MR methods does not require a contrast agent, but uses magnetic tagging of blood, e.g., by applying magnetically inverted spins in blood in the neck for use as an “intrinsic contrast agent” that is commonly referred to as “arterial spin labelling” (ASL). In ASL, blood with the tagged spins perfuses into an anatomical region of interest (ROI), where it is measured with fast MR imaging methods. In ASL, images are most often acquired with a perfusion-sensitive preparation (or tag) image and non-perfusion-sensitive (or control) image. Pulsed ASL (PASL) methods use a short inversion pulse for tagging or labeling the blood spins. For example, a 10 cm inversion slice is placed in the neck region and image slices are acquired from parts of the brain tissue.

The quality and value of ASL images depend heavily on the quality of the slice profile of the inversion pulse. Some of the important characteristics of this slice profile are efficiency of the inversion, edge steepness and amount of sidebands, pulse duration and amount of energy required for a full inversion pulse. Up to now, various types of pulses have been used to optimize the slice profile. However, the resulting ASL sequences still suffer from the fact that the pulses do not satisfy all needs in all situations.

ASL imaging is increasingly used in scanners with B₀ fields, scanners with larger bore sizes and scanners with low power radio frequency power amplifiers (RFPAs). However, such scanners present some challenges. Higher B₀ fields are usually accompanied by decreased homogeneity of the B₁ field such that a homogeneous inversion across the inversion slice becomes more challenging. This problem can be addressed by either increasing the pulse power or by choosing pulses which are less sensitive towards B₁ variations. Increasing the power is usually the less favorable option, since the specific absorption rate (SAR) increases and power in general is a limitation for the pulses.

Larger bore sizes (e.g., 70 cm vs. 60 cm) require the body coil to create the excitation field in a larger volume, which results in a higher power consumption of the RFPA while the effect of the pulses is the same. ASL sequences usually use adiabatic inversion pulses that need to be played out with a certain minimum amount of energy to achieve full inversion (or adiabatic condition). If the pulse energy is smaller than this threshold level, the inversion efficiency and the ASL signal is reduced. With the development towards larger bore sizes, the energy threshold level increases (due to the larger volume to be covered) and it becomes more and more challenging to meet this requirement.

ASL inversion pulses are in general very power consuming and can be limited by the RFPA power. It can be observed that more and more MR scanners are made available with “design to cost” RFPAs. These RFPAs are tailored to cover most of the standard MR applications. However, ASL is still in the transition between research and clinical use, and is therefore seldom considered in the “design to cost” process. For this reason, a reduction of the required pulse power—while maintaining the quality of the inversion profile—is desirable.

### SUMMARY

The present disclosure relates to a framework for facilitating low power perfusion imaging. In accordance with one aspect, a gradient-modulated constant-adiabaticity radio frequency pulse sequence is generated. A labeling pulse from the pulse sequence is applied to a region of interest to label fluid supplying the region of interest and to acquire a tagged image. A control pulse from the pulse sequence may also be applied to the region of interest to acquire a control image. A perfusion image may then be generated using the tagged image and the control image.

This summary is provided to introduce a selection of concepts in a simplified form that are further described below in the following detailed description. It is not intended to identify features or essential features of the claimed subject matter, nor is it intended that it be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings. Furthermore, it should be noted that the same numbers are used throughout the drawings to reference like elements and features.

FIG. 1 shows various exemplary waveforms of a frequency offset corrected inversion (FOCI) pulse;

FIG. 2 shows various exemplary waveforms of a gradient-modulated offset independent adiabaticity (GOIA) pulse;

FIG. 3 shows exemplary slice profiles of FOCI pulses at three different energy levels;

FIG. 4 shows exemplary slice profiles of GOIA pulses at three different energy levels;

FIG. 5 shows a comparison of exemplary FOCI and GOIA images associated with a first subject;

FIG. 6 shows a comparison of exemplary FOCI and GOIA images associated with a second subject;

FIG. 7 shows an exemplary plot of relative intensity curves based on a forty-slice ASL measurement;

FIG. 8 shows an exemplary low power ASL imaging system; and

FIG. 9 shows an exemplary low power perfusion imaging method.

### DETAILED DESCRIPTION

In the following description, numerous specific details are set forth such as examples of specific components, devices, methods, etc., in order to provide a thorough understanding of embodiments of the present invention. It will be apparent, however, to one skilled in the art that these specific details need not be employed to practice embodiments of the present invention. In other instances, well-known materials or methods have not been described in detail in order to avoid unnecessarily obscuring embodiments of the present invention. While the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present framework. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between” versus “directly between,” “adjacent” versus “directly adjacent,” etc.).

The present framework provides a system that facilitates low power perfusion imaging by using gradient-modulated low-power adiabatic RF pulses, such as a gradient-modulated offset independent adiabaticity (GOIA) pulse sequence. The gradient-modulated low-power adiabatic RF pulse can be scaled in duration and adiabatic frequency sweep to adjust the pulse properties according to the RFPA limits. The GOIA pulse can be used for blood inversion (labeling or control) and/or for other magnetization inversions (e.g., for background suppression).

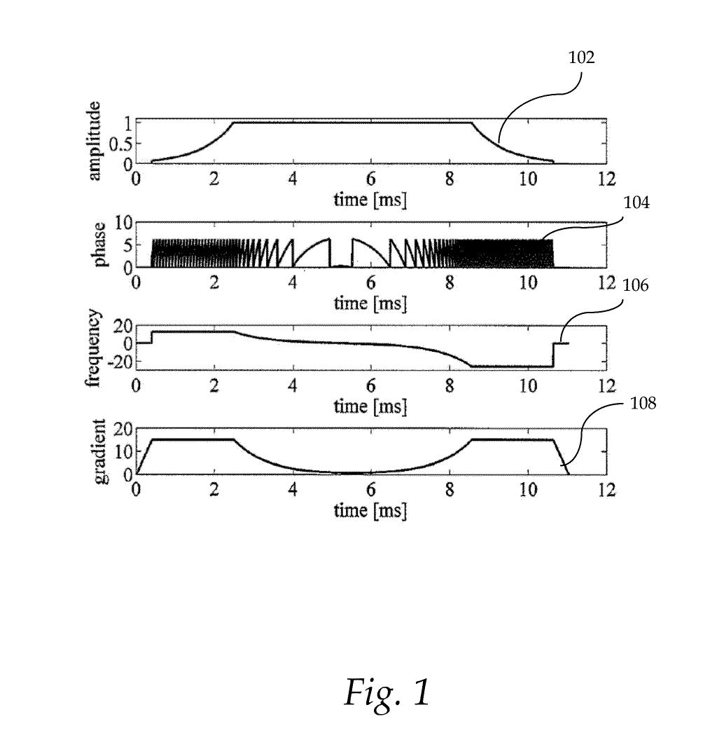

Traditional ASL product and research sequences typically use frequency offset corrected inversion (FOCI) pulses. FIG. 1 shows various exemplary waveforms of a FOCI pulse. More particularly, an amplitude waveform 102, phase waveform 104, a frequency waveform 106 and a gradient waveform 108 are shown. FOCI pulses utilize a variable rate selective excitation (VERSE) gradient which is non-constant in time. The FOCI pulse provides very high edge steepness and small amounts of sidebands. If the pulse energy threshold is met or exceeded, the inversion efficiency is very high across the whole slice profile (>97%). In case the pulse energy goes below threshold, the inversion efficiency is high at the edges of the slice but drops significantly in the center of the slice. The pulse duration is on the order of 10 ms. Much shorter pulses require higher power and often are limited by the RFPA specifications. Much longer pulses show relaxation effects of the spins during the excitation, and therefore have reduced inversion efficiency.

The power limit of RFPAs is usually incorporated in the calculation of the pulse properties. In case an inversion pulse cannot be realized within the RFPA limits, the following measures may be taken: The voltage of the pulse can be scaled down (often causing the pulse power to drop below the threshold) or the pulse can be scaled in time (often decreasing the inversion efficiency). Both solutions effectively result in a lower inversion efficiency and therefore decreased signal to noise ratio (SNR) in the ASL images.

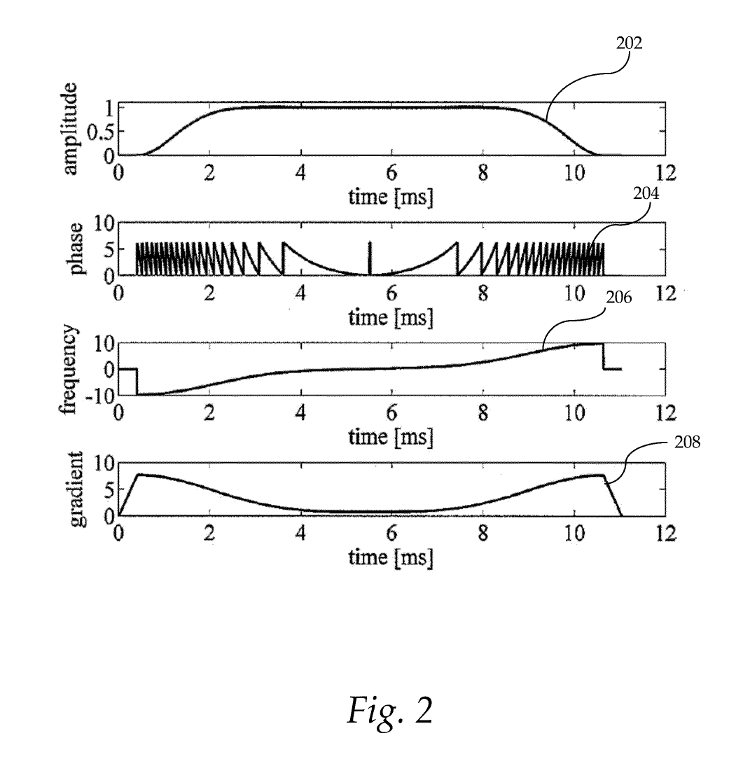

FIG. 2 shows various exemplary waveforms of a GOIA pulse. More particularly, an amplitude waveform 202, phase waveform 204, a frequency waveform 206 and a gradient waveform 208 of a GOIA pulse are shown. The GOIA pulse has a different amplitude and frequency modulation than conventionally used pulses (e.g., frequency offset correct inversion or FOCI). The GOIA pulse provides very high edge steepness and small amounts of sidebands. The same quality of edge steepness and sidebands, like in other pulses, with higher power requirements can be achieved. GOIA pulses require a lower energy threshold to achieve full inversion. Therefore, it is easier to fulfill the adiabatic condition. Calculations show that a GOIA pulse requires approximately only 60% of the energy threshold of a FOCI pulse to achieve full inversion. This increases the range of situations in which the pulses can be used without a quality penalty. Due to the lower power requirements of GOIA pulses, it is possible to shorten the pulses without exceeding RFPA limits. This might be advantageous in situations where the spin excitation needs to be very fast (e.g., movement of the spins or short relaxation times).

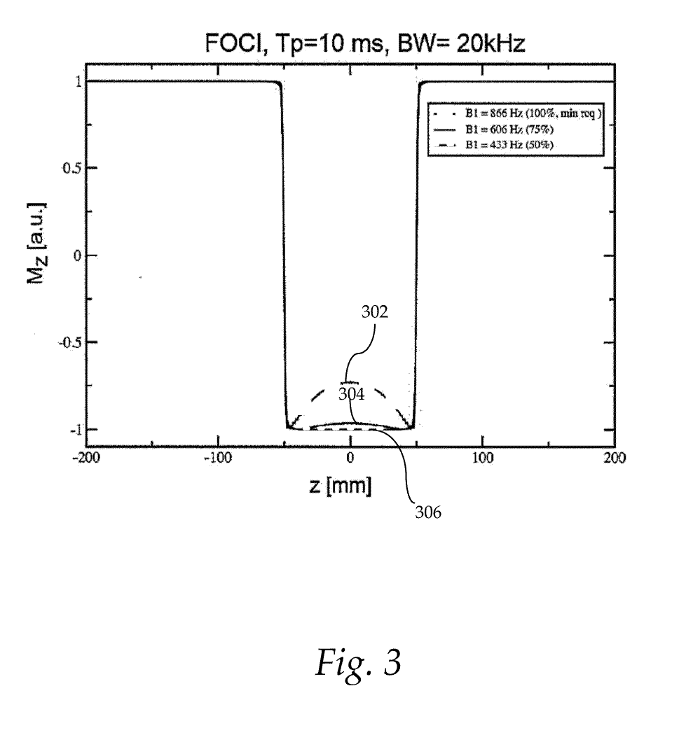

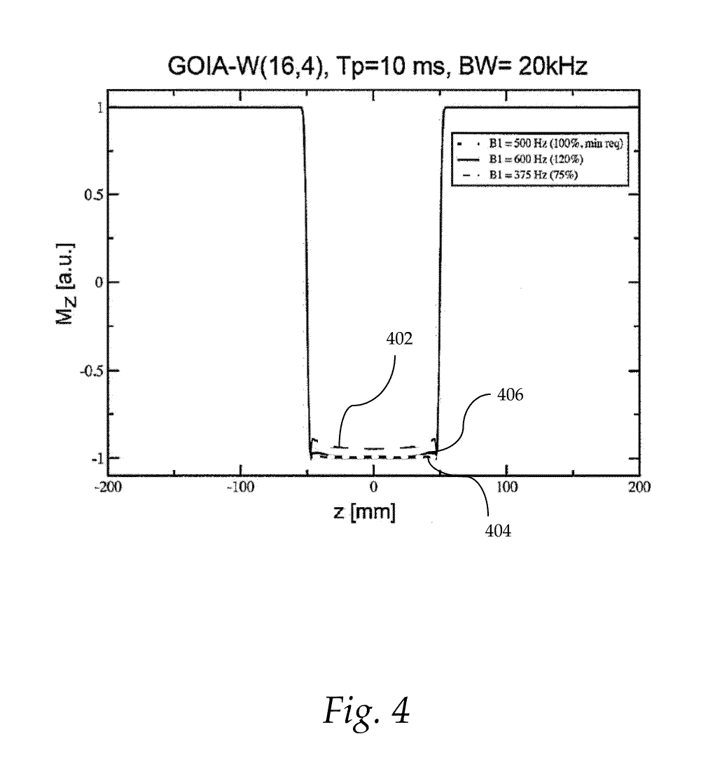

FIG. 3 shows exemplary slice profiles of FOCI pulses and FIG. 4 shows exemplary slice profiles of GOIA pulses at three different energy levels. Slice profiles 302 and 402 correspond to FOCI and GOIA pulses respectively with 100% threshold energy level. Slice profiles 304 and 404 correspond to FOCI and GOIA pulses respectively with 75% of the threshold energy level. Slice profiles 306 and 406 correspond to FOCI and GOIA pulses with 50% and 120% of the threshold energy level respectively.

The GOIA pulses produce a more homogeneous slice profile than the FOCI pulses if they are used below the energy threshold (or adiabatic condition). In some cases, it may not be possible to meet the threshold condition (e.g., due to RFPA limits or B₁ inhomogeneity). Nonetheless, the GOIA pulses show a very good natured behavior. They have a global decrease of inversion efficiency (homogeneous across the whole slice profile). In comparison, FOCI pulses show a much more inhomogeneous inversion efficiency across the slice profile in this situation. This leads to a more homogeneous image intensity which is less sensitive to B₁ variations.

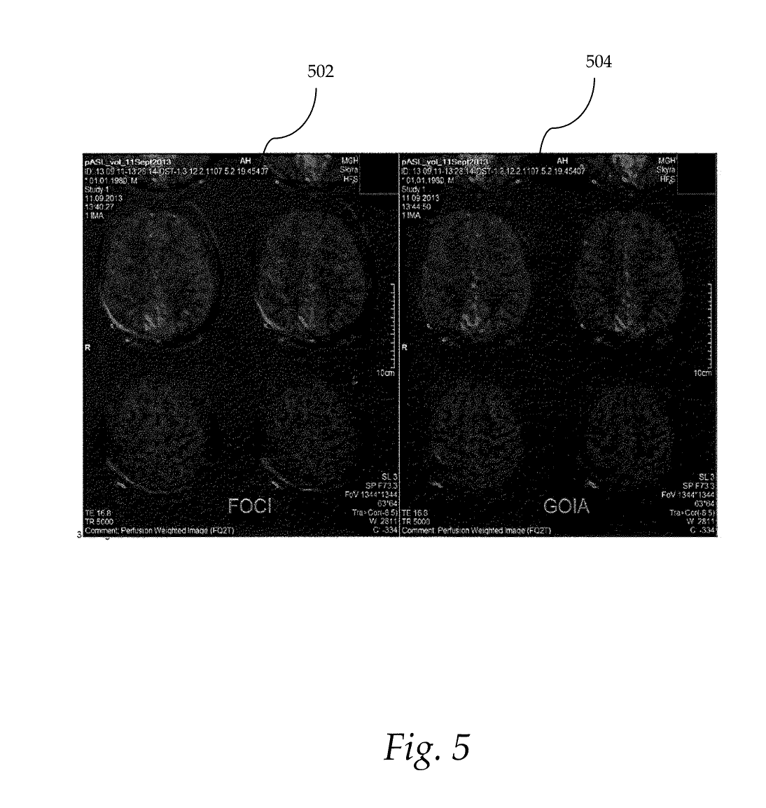

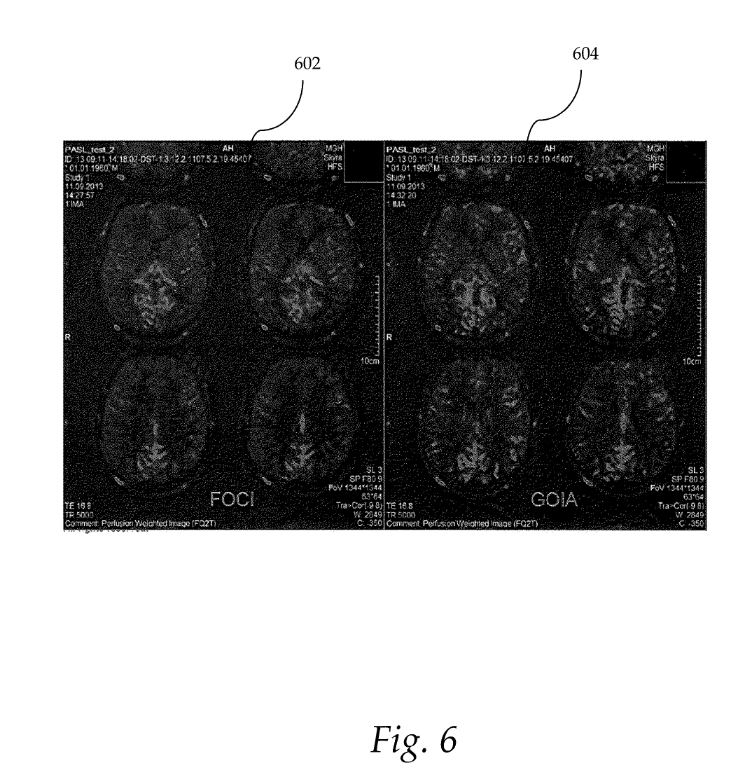

FIG. 5 shows a comparison of FOCI and GOIA images (502 and 504 respectively) associated with a first subject, while FIG. 6 shows a comparison of FOCI and GOIA images (602 and 604 respectively) associated with a second subject. It can be observed that the FOCI pulse based perfusion signal (as shown in images 502 and 602) is lower compared to GOIA pulse based perfusion signal (as shown in images 504 and 604). This is due to the FOCI pulse power being limited by the RFPA, while the GOIA pulse power was not limited. The GOIA pulse data (in images 504 and 604) shows increased signal in both comparisons across both subjects.

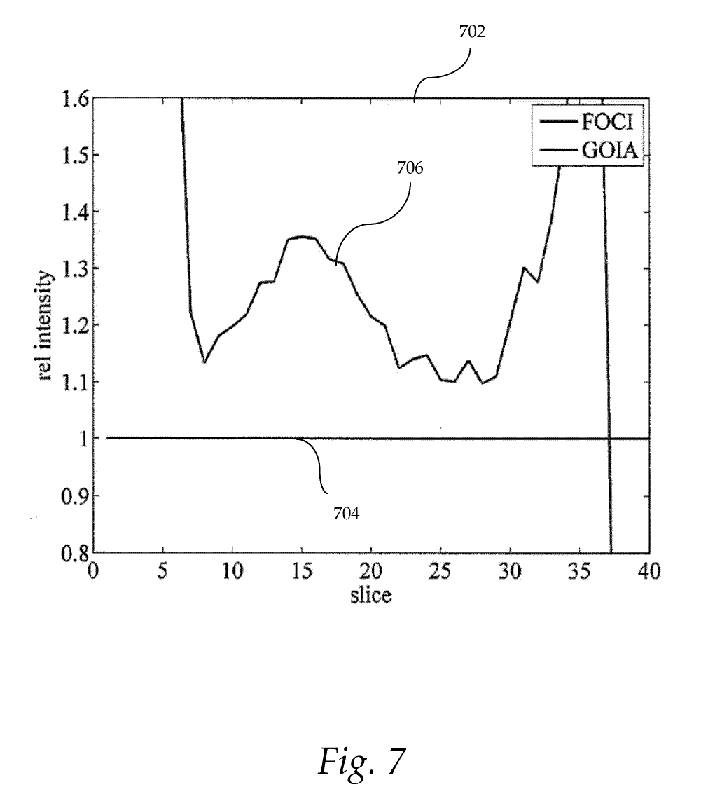

FIG. 7 shows a plot 702 of relative intensity curves (704 and 706) based on a forty-slice ASL measurement. The global slice intensities were normalized to the FOCI data associated with relative intensity curve 704. The relative intensity curve 706 associated with the GOIA data shows a signal increase of about 20% for the most important center slices (measured as the region of interest over the whole head).

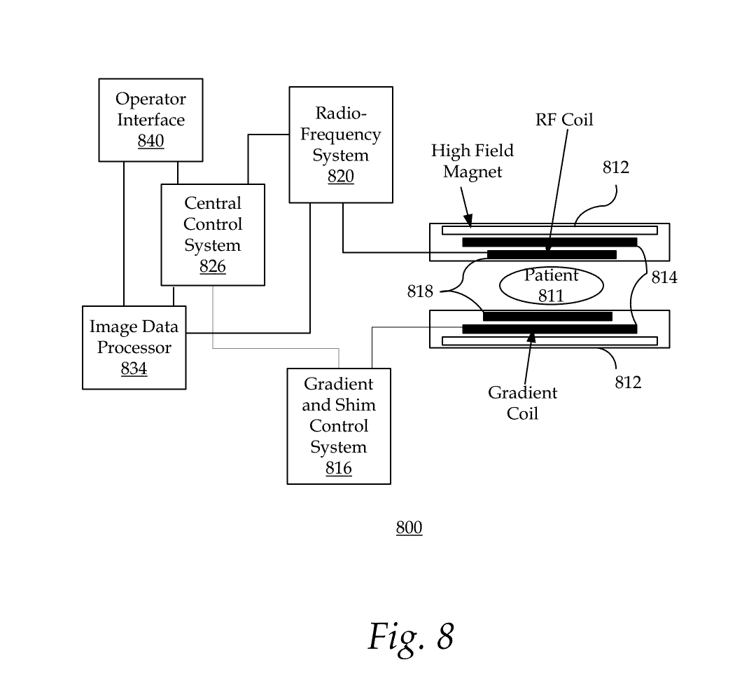

FIG. 8 shows an exemplary low power ASL imaging system 800 that employs GOIA pulse sequences. In system 800, magnet 812 creates a static base magnetic field (B₀) in the body of patient 811 to be imaged and positioned on a table. Within the magnet system are gradient coils 814 for producing position dependent magnetic field gradients superimposed on the static magnetic field. Gradient coils 814, in response to gradient signals supplied thereto by a gradient module 816, produce position dependent and shimmed magnetic field gradients in three orthogonal directions and generate magnetic field pulse sequences. The shimmed gradients compensate for inhomogeneity and variability in an MR imaging device magnetic field resulting from patient anatomical variation and other sources. The magnetic field gradients include a slice-selection gradient magnetic field, a phase-encoding gradient magnetic field and a readout gradient magnetic field that are applied to patient 811.

Further, radio frequency (RF) system 820 provides RF pulse signals to RF coil 818, which in response produces magnetic field pulses which rotate the spins of the protons in the imaged body 811 by ninety degrees or by one hundred and eighty degrees for so-called “spin echo” imaging, or by angles less than or equal to 90 degrees for gradient echo imaging. Pulse sequence control system 816, in conjunction with RF system 820 as directed by central control system 826, control slice-selection, phase-encoding, readout gradient magnetic fields, radio frequency transmission, and magnetic resonance signal detection, to acquire magnetic resonance signals representing planar slices of patient 811.

In response to applied RF pulse signals, the RF coil 818 receives MR signals. For example, RF coils 818 may receive signals from the excited protons within the body as they return to an equilibrium position established by the static and gradient magnetic fields. MR signals are detected and processed by a receiver within RF system 820 to generate image representative data to an image data processor 834. RF system 820 further includes a generator that generates an RF excitation pulse sequence having a pulse repetition interval. Read-out gradient magnetic field generator 814 generates a read-out gradient magnetic field, in response to gradient signals supplied by gradient system 816. The read-out gradient magnetic field is used in acquiring multiple individual frequency components and generates magnetic field gradients for anatomical slice selection, phase encoding and readout RF data acquisition in a three dimensional (3D) anatomical volume.

A data acquisition device or receiver in RF system 820 acquires RF echo data generated in response to the RF excitation pulse sequence and a controller in RF system 820 directs acquisition and processing of the RF echo data. MR signals detected and processed by a detector within RF system 820 provide image representative data to image data processor 834. Image data processor 834 acquires an anatomical imaging data set representing a slice of patient 811 anatomy. Image data processor 834 initiates acquisition of a first image set comprising multiple different individual images having a set of corresponding different physical slice locations through a patient anatomical volume and being acquired at a corresponding first set of times and in a first order relative to a time of blood tagging of a patient. Image data processor 834 initiates acquisition of a second image set comprising multiple different individual images having the set of corresponding different physical slice locations through the patient anatomical volume and that are acquired at substantially the corresponding first set of times and in a second order, different to the first order, relative to the time of blood tagging of the patient. Image data processor 834 comprises at least one processing device that combines and averages image data representing the same corresponding image slice in both the first and second image sets and acquired at different times relative to the time of blood tagging of the patient. Image data processor 834 sends the combined and averaged image data representing the same corresponding image slice to a destination.

A display processor in operator interface computer 840 generates data representing at least one two-dimensional display image using the combined and averaged image data. Central control unit 826 uses information stored in an internal database comprising predetermined pulse sequence and strength data as well as data indicating timing, orientation and spatial volume of gradient magnetic fields to be applied in imaging and adjusts other parameters of system 800, so as to process the detected MR signals in a coordinated manner to generate high quality images of a selected slice (or slices) of the body. Generated images are presented on a display device at operator interface computer 840. Operator interface computer 840 may include a graphical user interface (GUI) enabling user interaction with central controller 826 and enabling user modification of magnetic resonance imaging signals in substantially real time. A data acquisition device in unit 820, in conjunction with image data processor 834 and operator interface computer 840, processes the magnetic resonance signals to provide image representative data for display on, for example, operator interface computer 840.

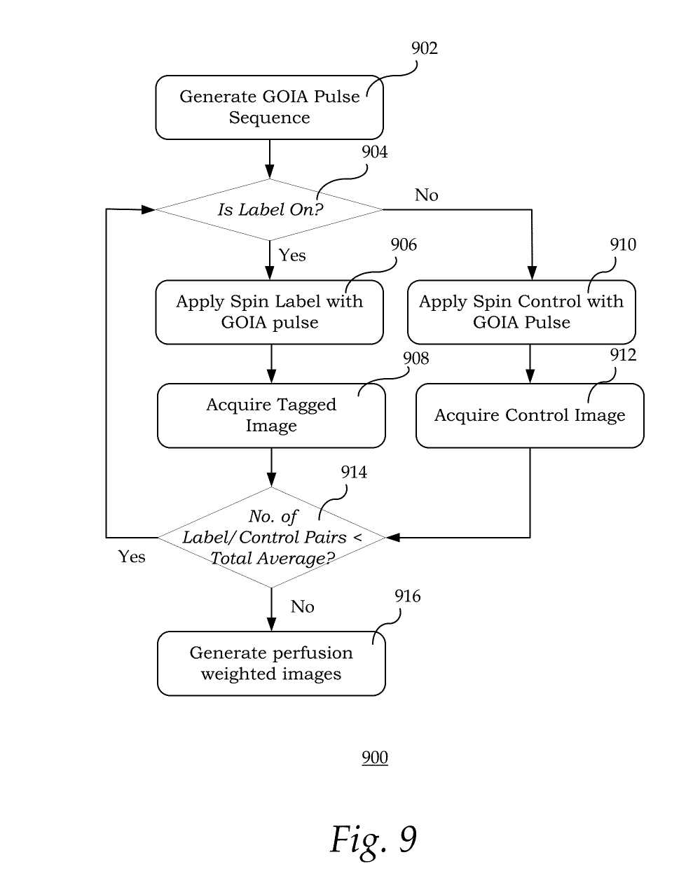

FIG. 9 shows an exemplary low power perfusion imaging method 900. The steps of the method 900 may be performed in the order shown or a different order. Additional, different, or fewer steps may be provided. Further, the method 900 may be implemented with the system 800 of FIG. 8, a different system, or a combination thereof.

At 902, RF system 820 initializes or generates gradient-modulated constant-adiabaticity RF pulse sequence, such as Gradient Offset Independent Adiabaticity (GOIA) pulses with, for example, WURST modulations (e.g., GOIA-W(16, 4) pulses) or hyperbolic secant modulations (e.g., GOIA-HS(8,4) pulses). As described previously, GOIA pulses are favorable because they produce more homogeneous slice profiles and require a lower energy threshold to achieve full inversion and fulfill the adiabatic condition. Due to the lower power requirements of GOIA pulses, it is possible to shorten the pulses without exceeding RFPA limits. Pulse sequence control system 816 may scale the GOIA pulse in duration and adiabatic frequency sweep to adjust pulse properties according to RFPA limits. For example, the GOIA pulse may be scaled to a pulse duration of 10 ms and frequency sweep of 20 kHz. The GOIA pulse may then be used for blood inversion (labeling or control) and/or for other magnetization inversions (e.g., for background suppression).

At 904, RF system 820 determines if a “label” parameter is on. If the “label” parameter is on, at 906, RF system 820 applies a labeling pulse from the pulse sequence to a region of interest to tag or label fluid (e.g., blood) by inversion. The region of interest may be an anatomical region or volume of the patient that has been identified for further study, such as a portion of the neck or brain. In some implementations, pulsed arterial spin labeling (ASL) is performed to magnetically tag the fluid before it enters the region of interest. Pulsed ASL involves applying an RF labeling pulse (e.g., GOIA pulse) to invert or saturate water protons in flowing fluid (i.e., arterial spins) supplying the region of interest. In some implementations, one or more separate inversion pulses derived from the pulse sequence are applied to the fluid after placing the labeling pulse to achieve a high level of background suppression to reduce motion and other sources of noise.

At 906, RF system 820 initiates acquisition of the tagged (or label-on) image. In some implementations, a two-dimensional (2D) or three-dimensional (3D) MR tagged image representative data is acquired.

If the “label” parameter is off, at 910, RF system 820 initiates a control procedure by applying a control pulse from the pulse sequence. The control pulse does not cause significant inversion of the water protons in the region of interest. The fluid in the region of interest is not labeled but all other parameters remain unchanged so as to provide a measure of tissue perfusion of the labeled fluid with subtraction of effects by other sources of signal that are constant in the two label/control image acquisitions.

At 912, RF system 820 initiates acquisition of the control (or label-off) image. The control image is non-perfusion-sensitive and is acquired in a manner similar to the tagged image.

At 914, RF system 820 determines if the number of pairs of label and control images is less than a total average. If yes, the method 900 returns at 904 to acquire additional tagged (or label-on) and/or control (or label-off) images. Accordingly, a set of corresponding tagged and control image pairs are acquired according to a pre-determined label-on/label-off sequence. In some implementations, the user may be presented, via a user interface, with a choice of the type of label-on/label-off sequence to be employed.

If the number of pairs of label/control images is more than a total average, the method 900 continues at 916 to generate perfusion weighted images based on the pairs of corresponding label and control images. The perfusion images may be generated in real-time and updated during the process of an imaging examination of the patient, or performed offline after the imaging examination. Image data processor 834 may generate perfusion weighted images by differentially combining or subtracting the control image from the tagged image of each pair of corresponding images. Appropriate scaling factors may be applied.

A processor, as used herein, is a device for executing machine-readable instructions stored on a non-transitory, tangible computer readable medium, for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a computer, controller or microprocessor, for example, and is conditioned using executable instructions to perform special purpose functions not performed by a general purpose computer. A processor may be coupled (electrically and/or as comprising executable components) with any other processor or system enabling interaction and/or communication there-between. A user interface processor or generator is a known element comprising electronic circuitry or software or a combination of both for generating display images or portions thereof. A user interface comprises one or more display images enabling user interaction with a processor or other device.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A graphical user interface (GUI), as used herein, comprises one or more display images, generated by a display processor and enabling user interaction with a processor or other device and associated data acquisition and processing functions.

The user interface (UI) also includes an executable procedure or executable application. The executable procedure or executable application conditions the display processor to generate signals representing the UI display images. These signals are supplied to a display device which displays the image for viewing by the user. The executable procedure or executable application further receives signals from user input devices, such as a keyboard, mouse, light pen, touch screen or any other means allowing a user to provide data to a processor. The processor, under control of an executable procedure or executable application, manipulates the UI display images in response to signals received from the input devices. In this way, the user interacts with the display image using the input devices, enabling user interaction with the processor or other device. The functions and process steps herein may be performed automatically or wholly or partially in response to user command. An activity (including a step) performed automatically is performed in response to executable instruction or device operation without user direct initiation of the activity.

While the present invention has been described in detail with reference to exemplary embodiments, those skilled in the art will appreciate that various modifications and substitutions can be made thereto without departing from the spirit and scope of the invention as set forth in the appended claims. For example, elements and/or features of different exemplary embodiments may be combined with each other and/or substituted for each other within the scope of this disclosure and appended claims.

1. A low power perfusion imaging system, comprising:
a radio frequency signal system configured to
generate a gradient-modulated constant-adiabaticity radio frequency pulse sequence,
apply a labeling pulse from the pulse sequence to a region of interest to label fluid supplying the region of interest and to acquire a tagged image,
apply a control pulse from the pulse sequence to the region of interest to acquire a control image; and
an image data processor configured to generate a perfusion image using the tagged image and the control image.

2. The system of claim 1 wherein the fluid is blood in a portion of a brain.

3. The system of claim 1 wherein the gradient-modulated constant-adiabaticity RF pulse sequence comprises Gradient Offset Independent Adiabaticity (GOIA) pulses.

4. The system of claim 3 wherein the GOIA pulses comprise GOIA pulses with WURST modulations.

5. The system of claim 3 wherein the GOIA pulses comprise GOIA pulses with hyperbolic secant modulations.

6. The system of claim 1 wherein the radio frequency signal system configured to label fluid in the region of interest by arterial spin labeling.

7. The system of claim 1 wherein the radio frequency signal system configured to apply one or more inversion pulses from the pulse sequence to the region of interest for background suppression.

8. The system of claim 1 wherein the tagged image and the control image comprise magnetic resonance images.

9. The system of claim 1 wherein the image data processor is configured to generate the perfusion image by subtracting the control image from the tagged image.

10. A low power perfusion imaging method, comprising:
generating, via a radio frequency signal system, a gradient-modulated constant-adiabaticity radio frequency pulse sequence;
applying, by the radio frequency signal system, a labeling pulse from the pulse sequence to a region of interest to label fluid supplying the region of interest;
acquiring a tagged image of the region of interest with the labeled fluid;
applying, by the radio frequency signal system, a control pulse from the pulse sequence to the region of interest;
acquiring a control image of the region of interest; and
generating, by an image data processor, a perfusion image using the tagged image and the control image.

11. The method of claim 10 wherein the fluid is blood in a portion of a brain.

12. The method of claim 10 wherein applying the gradient-modulated constant-adiabaticity RF pulse sequence comprises applying Gradient Offset Independent Adiabaticity (GOIA) pulses.

13. The method of claim 12 wherein applying the GOIA pulses comprises applying GOIA pulses with WURST modulations.

14. The method of claim 12 wherein applying the GOIA pulses comprises applying GOIA pulses with hyperbolic secant modulations.

15. The method of claim 10 wherein applying the labeling pulse comprises performing arterial spin labeling using the labeling pulse.

16. The method of claim 10 further comprising applying one or more inversion pulses from the pulse sequence to the region of interest for background suppression.

17. The method of claim 10 wherein acquiring the tagged image and the control image comprises acquiring magnetic resonance images.

18. The method of claim 10 wherein generating the perfusion image comprises subtracting the control image from the tagged image.
